(19) United States
(12) Patent Application Publication
(10) Pub. No.: US 2025/0319013 A1
(43) Pub. Date: Oct. 16, 2025

(54) THICKENING COMPOSITION FOR THICKENING POLAR OR NON-POLAR OILS

(71) Applicant: SOCIETE D'EXPLOITATION DE PRODUITS POUR LES INDUSTRIES CHIMIQUES SEPPIC., PARIS CEDEX 07 (FR)

(72) Inventors: Miruna BODOC, CASTRES (FR); Estelle ILLOUS, CASTRES (FR); Virginie BARTHE, CASTRES (FR); Georges Manuel DA COSTA, CASTRES (FR); Jerome GUILBOT, CASTRES (FR)

(21) Appl. No.: 18/572,726

(22) PCT Filed: Jun. 22, 2022
(86) PCT No.: PCT/EP2022/067059

§ 371 Date: Dec. 20, 2023

(30) Foreign Application Priority Data
Jun. 23, 2021 (FR) FR2106665

(51) Int. Cl. A61K 8/81; A61K 8/92; A61Q 19/00

(52) U.S. Cl. CPC A61K 8/8164; A61K 8/92; A61Q 19/00; A61K 2800/48

(57) ABSTRACT

The invention relates to a thickening composition for thickening polar or non-polar oils, including at least an oil and a poly.

### CROSS-REFERENCE TO RELATED APPLICATIONS

This application is the US national stage of PCT/EP2022/067059, filed Jun. 22, 2022 and designating the United States, which claims the priority of FR2106665, filed Jun. 23, 2021. The entire contents of each foregoing application are incorporated herein by reference.

### BACKGROUND OF THE INVENTION

### Field of the Invention

The present invention relates to a composition for thickening polar or apolar oils, to the process for manufacturing same and to topical formulations comprising this thickening composition.

### Description of the Related Art

There is a need to thicken oils in the cosmetics market. Gel formation requires the formation of a three-dimensional network by a gelling agent, enabling immobilization of the liquid phase or the solvent. Several strategies may be used to do this. They involve various types of interactions, including weak ones (hydrogen bonding, hydrophobic/hydrophobic, etc.) and absorption/adsorption phenomena.

The thickening solutions currently proposed offer significant performance and appreciable advantages in terms of versatility, but also drawbacks, notably the impact on the formula's sensory properties, and the formulation conditions. These substances are of varied chemical nature: silica derivatives, clays, polymers such as polyamide, polyacrylate, polyurethane, beeswax, hydrogenated plant oils or else sugar fatty esters or amides.

Although there are already oil thickeners on the market, of varied nature and performance, the relationships between their structures and properties are not clearly identified, which is the main scientific barrier, since the object is to find a structure obtained from materials of plant origin which would afford high performance with oils of any type of nature (polar to nonpolar), for different types of textures (compact to fluid) and attractive or at least non-prohibitive sensory properties (non-greasy, non-tacky, non-runny).

One of the difficulties thus lies in finding a single universal structure or substance allowing all types of oils, from the most apolar to the most polar, to be effectively thickened at low doses, the gelling mechanisms of course differing according to the nature of the oil and the gelling agent.

### SUMMARY OF THE INVENTION

A solution of the present invention is a composition (C) for thickening polar and/or apolar oils (H2), comprising at least one oil (H1) and a poly(itaconate) (P).

Said thickening composition (C) makes it possible to thicken polar oils (H2) and apolar oils.

For the purposes of the present invention, the term “polar oil” denotes a hydrophobic compound which comprises polar groups in its molecular structure, i.e. groups that are capable of creating electrostatic interactions with water molecules, of the permanent dipole/permanent dipole type.

For the purposes of the present invention, the term “apolar oil” denotes a hydrophobic compound which cannot create electrostatic interactions with water molecules of the permanent dipole/permanent dipole type.

For the purposes of the present invention, the term “hydrophobic compound” denotes a compound which cannot physically interact with water.

For the purposes of the present invention, the term “oil” denotes a compound and/or a mixture of compounds that are hydrophobic and insoluble in water.

Preferably, the poly(itaconate) (P) will consist:
of at least one monomer unit derived from itaconic acid of formula (I) in free acid form or partially or totally salified form

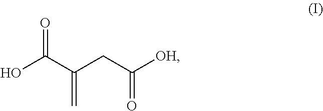

and
of at least one monomer unit derived from a compound of formula (II):

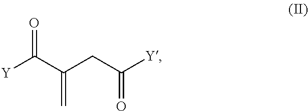

with Y and Y′, which are identical, representing the radical —OR₁, or Y and Y′, which are different, representing either the —OH radical or the radical —OR₁, with R₁ representing:
a) a linear or branched, saturated or unsaturated, aliphatic hydrocarbon-based radical (IIa) including from 6 to 36 carbon atoms and optionally substituted with one or more hydroxyl groups,
or
b) a radical of formula (IIb) in which R₂ represents a linear or branched, saturated or unsaturated, aliphatic hydrocarbon-based radical including from 6 to 36 carbon atoms and optionally substituted with one or more hydroxyl groups:

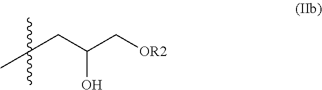

or
c) a radical of formula (IIc) in which R₃—C(═O) represents a linear or branched, saturated or unsaturated hydrocarbon-based acyl radical containing from 12 to 24 carbon atoms, and more particularly a radical chosen from the elements of the group consisting of dodecanoyl, tetradecanoyl, hexadecanoyl, octadecanoyl, eicosanoyl, docosanoyl, oleyl, linoleyl, linolenoyl or isostearyl radicals, and in which x represents a natural integer greater than or equal to 1 and less than or equal to 6:

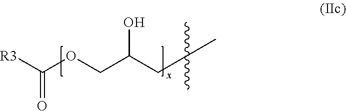

or
d) a radical of formula (IId) in which m represents an integer greater than or equal to 2 and less than or equal to 10:

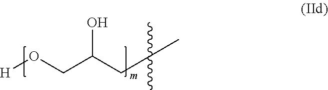

or
e) a radical of formula (lie) in which o represents an integer greater than or equal to 2 and less than or equal to 4:

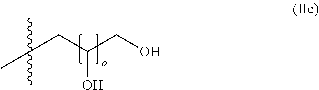

or
f) a radical of formula (IIf):

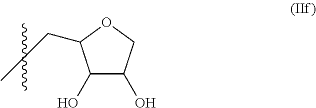

or
g) a radical of formula (IIg):

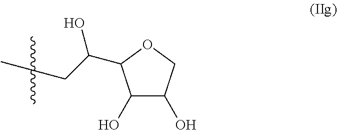

or
h) a radical of formula (IIh) in which q represents an integer greater than or equal to 1 and less than or equal to 12, more particularly greater than or equal to 3 and less than or equal to 8:

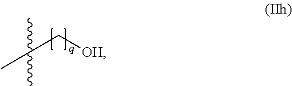

when Y and Y′ represent the —OH radical, the carboxylic acid function then present in formula (II) is in free acid form or partially or totally salified form.

For the purposes of the present invention, the term “salified” means that the acid function present in a monomer is in an anionic form associated in salt form with a cation, notably alkali metal salts, such as the sodium or potassium cations, or such as cations of nitrogenous bases such as the ammonium salt, the lysine salt or the monoethanolamine salt (HOCH₂—CH₂—NH₄⁺). They are preferably sodium or ammonium salts.

The term “saturated or unsaturated, linear or branched aliphatic hydrocarbon-based radical including from 6 to 36 carbon atoms, optionally substituted with one or more hydroxyl groups” denotes, for the radical R₁ of formula (IIa) and for the radical R₂ of formula (IIb) as defined above:
saturated linear alkyl radicals, for example n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl, n-eicosyl and n-docosyl radicals;
unsaturated linear radicals such as octenyl, decenyl, undecenyl, dodecenyl, tridecenyl, tetradecenyl, pentadecenyl, hexadecenyl, heptadecenyl, octadecenyl, nonadecenyl, eicosenyl, docosenyl, 4-dodecenyl and 5-dodecenyl radicals;
saturated or unsaturated, linear or branched aliphatic radicals including from 6 to 36 carbon atoms substituted with one or two hydroxyl groups, such as hydroxydodecyl, hydroxytetradecyl, hydroxyhexadecyl, hydroxyoctadecyl, hydroxyeicosyl and hydroxydocosyl radicals, for example the 12-hydroxyoctadecyl radical;
radicals derived from the isoalkanols of formula (1):

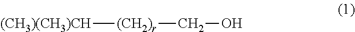

in which r represents an integer between 2 and 20, for example the isodecyl, isoundecyl, isododecyl, isotridecyl, isotetradecyl, isopentadecyl, isohexadecyl, isoheptadecyl, isooctadecyl, isononadecyl, isoeicosyl or isodocosyl radicals;
branched alkyl radicals, derived from Guerbet alcohols, of formula (2):

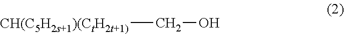

in which t is an integer between 4 and 18, s′ is an integer between 2 and 18 and the sum s+t is greater than or equal to 6 and less than or equal to 22, for example the 2-butyloctyl, 2-butyldecyl, 2-hexyloctyl, 2-hexyldecyl, 2-octyldecyl, 2-hexyldodecyl, 2-octyldodecyl, 2-decyltetradecyl, 2-dodecylhexadecyl or 2-tetradecyloctadecyl radicals.

According to an even more particular aspect, in the definition of formulae (IIa) and (IIb) as defined above, R₁ and R₂ represent a radical chosen from at least one of the elements of the group consisting of n-hexyl, n-heptyl, n-octyl, n-nonyl, n-decyl, n-dodecyl, n-tetradecyl, n-hexadecyl, n-octadecyl, n-eicosyl, n-docosyl, 2-hexyldecyl, 2-octyldecyl, 2-hexyldodecyl, 2-octyldodecyl, 2-decyltetradecyl and 12-hydroxyoctadecyl radicals.

According to a particular aspect, the poly(itaconate) (P) is a crosslinked poly(itaconate) (P).

For the purposes of the present invention, the term “crosslinked poly(itaconate) (P)” denotes poly(itaconate) (P) as defined previously and also consisting of at least one monomer unit derived from a polyethylenic crosslinking monomer (AR).

The term “polyethylenic crosslinking monomer (AR)” denotes compounds whose chemical formula includes at least two ethylenic bonds and which allow two or more polymer chains to be linked during the radical polymerization reaction involving the monomers making up the poly(itaconate) as described above. The polyethylenic crosslinking monomers (AR) may be added to the monomers prior to initiation of the radical polymerization process allowing the crosslinked poly(itaconate) (P) to be prepared.

The polyethylenic crosslinking monomers (AR) may also be constituents of the monomer compositions used in the radical polymerization process, as a result of the esterification between two or more itaconic acid molecules with one molecule of a polyol.

For the purposes of the present invention, the term “polyol” denotes an aliphatic molecule substituted with at least two hydroxyl groups.

Depending on the case, the solution according to the invention may have one or more of the following features:
composition (C) comprises between 20% and 80% by mass, preferably between 40% and 60% by mass, even more preferentially between 43% and 57% by mass of oil (H1) and between 80% and 20% by mass, preferably between 60% and 40% by mass, even more preferentially between 57% and 43% by mass of poly(itaconate) (P);
the oil (H1) is an oil that is in liquid form at a temperature of between 4° C. and 45° C., preferably between 15° C. and 45° C. and even more preferentially at a temperature of between 15° C. and 35° C.;
the oil (H1) is a mineral or plant oil or a mixture of these two types of oil;
the mass ratio (P)/(H1) is between 5/1 and 1/5, preferably between 4/1 and 1/4, more preferentially between 3/1 and 1/3, even more preferentially between 1/2 and 2/1.

The mineral oils are preferably chosen from:
paraffins, isoparaffins or cycloparaffins such as: mixtures of alkanes and isoalkanes and cycloalkanes sold under the names Emogreen™ L15, Emogreen™ L19, Emosmart™ L15, Emosmart™ L19, Emosmart™ V21, Isopar™ L, Isopar™ H, Isopar™ G or Isopar™ M,
white mineral oils, such as the products sold under the following names: Marcol™ 52, Marcol™ 82, Drakeol™ 6VR, Eolane™ 130, Eolane™ 150,
linear alkanes including from 11 to 19 carbon atoms, and more particularly n-undecane, n-dodecane, n-tridecane, and even more particularly the mixture of n-undecane and n-tridecane,
branched alkanes including from 7 to 20 carbon atoms, such as isododecane, isopentadecane, isohexadecane, isoheptadecane, isooctadecane, isononadecane or isoeicosane, or mixtures of some of them identified by their INCI name: C7-8 isoparaffin, C8-9 isoparaffin, C9-11 isoparaffin, C9-12 isoparaffin, C9-13 isoparaffin, C9-14 isoparaffin, C9-16 isoparaffin, C10-11 isoparaffin, C10-12 isoparaffin, C10-13 isoparaffin, C11-12 isoparaffin, C11-13 isoparaffin, C11-14 isoparaffin, C12-14 isoparaffin, C12-20 isoparaffin, C13-14 isoparaffin, C13-16 isoparaffin;
cycloalkanes optionally substituted with one or more linear or branched alkyl radicals;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene.

The plant oils are preferably chosen from oils belonging to the family:
of the lipophilic esters of formula (IV),

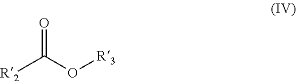

in which R′2—(C═O)— represents a saturated or unsaturated, linear or branched acyl radical including from 8 to 24 carbon atoms, and R′3 represents, independently of R′2, a saturated or unsaturated, linear or branched hydrocarbon-based chain including from 1 to 24 carbon atoms, the compound of formula (IV) representing, for example, methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, octyl cocoate, decyl cocoate, octyl and decyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate.

According to a particular aspect, the compound of formula (IV) represents cocoyl caprylate caprate such as the product sold under the brand name DUB 810C, propyl cocoate, isopropyl cocoate, propyl palmitate, isopropyl palmitate, octyl palmitate.
triglycerides of formula (V)

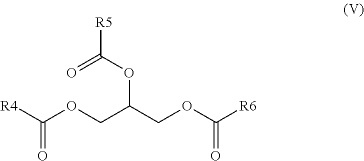

in which R4-(C═O), R5-(C═O) and R6-(C═O), which may be identical or different, represent a linear or branched, saturated or unsaturated acyl group containing from 8 to 24 carbon atoms, more particularly an acyl group chosen from the elements of the group consisting of octanoyl, decanoyl, dodecanoyl, tetradecanoyl, hexadecanoyl, octadecanoyl, eicosanoyl, docosanoyl, oleyl, linoleyl, linolenoyl or isostearyl groups.

The term “oil (H2)” means oils (H1) as defined previously, and more particularly mineral oils as described above and compounds of formula (V) in which R4-(C═O), R5-(C═O) and R6-(C═O), which may be identical or different, represent an acyl group chosen from the elements of the group consisting of octanoyl, decanoyl and dodecanoyl groups.

A subject of the present invention is also a topical formulation (F) comprising a composition (C) as defined previously. In other words, the topical formulation (F) comprises the mixture of composition (C) and at least one oil (H2).

The expression “for topical use” used in the definition of formulation (F) as defined above means that said formulation is prepared to allow its application to the skin, the hair, the scalp or the mucous membranes, whether it is direct application in the case of a cosmetic, dermocosmetic, dermopharmaceutical or pharmaceutical composition or an indirect application, for example in the case of a body hygiene product in the form of a textile or paper wipe, or sanitary products intended to be in contact with the skin or the mucous membranes.

Preferably, this topical formulation (F) will be a cosmetic formulation.

Said topical formulation (F) is generally in the form of an oily composition, in the form of a suspension, an emulsion, a microemulsion or a nanoemulsion, whether they are of water-in-oil, oil-in-water, water-in-oil-in-water or oil-in-water-in-oil type.

Said topical formulation (F) may be packaged in a bottle, in a device of pump-bottle type, in pressurized form in an aerosol device, in a device equipped with an openwork wall such as a grate or in a device equipped with a ball applicator (known as a roll-on).

In general, said topical composition (F) also includes excipients and/or active principles usually used in the field of formulations for topical use, in particular cosmetic, dermocosmetic, pharmaceutical or dermopharmaceutical formulations, such as thickening and/or gelling surfactants, stabilizers, film-forming compounds, hydrotropic agents, plasticizers, emulsifying and coemulsifying agents, opacifiers, nacreous agents, superfatting agents, sequestrants, chelating agents, antioxidants, fragrances, preserving agents, conditioning agents, bleaching agents intended for bleaching bodily hairs and the skin, active principles intended to provide a treating action with respect to the skin or the hair, sunscreens, pigments or mineral fillers, particles affording a visual effect or intended for the encapsulation of active principles, exfoliant particles or texturing agents.

The mixture of composition (C) as described previously and an oil (H2) will preferably be prepared at a temperature of between 50° C. and 85° C., more preferentially at a temperature of 80° C. The homogeneous oily composition obtained is then cooled to room temperature, between 15° C. and 30° C.

The thickening of the oil (H2) is finally assessed visually by qualifying the viscous or gelled liquid consistencies, by measuring a dynamic viscosity value.

For a composition (C) to be considered efficient, the viscous or gelled aspects must at least be achieved.

A subject of the present invention is also a process for preparing a thickening composition (C) according to the invention, comprising the following steps:
a) production of a composition (C0) of itaconic acid mono- and/or oligoesters comprising a compound of formula (II) and the compound of formula (1) in free acid form or partially or totally salified form,
b) preparation of a mixture (M1) of the composition (C0) produced in step a) with an oil (H1),
c) polymerization of the mixture (M1) initiated by introducing a thermal radical initiator into said mixture (M1) obtained on conclusion of step b), and
d) recovery of composition (C).

The term “thermal initiator” in step c) of the process as defined previously denotes a molecule which, when placed at a temperature specific to said structure, generates the production of at least two radicals by homolysis of at least one covalent bond, said radicals enabling the initiation of radical polymerization.

According to a particular aspect, the thermal initiator is chosen from the elements of the group consisting of lauroyl peroxide, azobis(isobutyronitrile) or AIBN, benzoyl peroxide or BPO, azobis((2-methylbutyronitrile) or AMBN, 4,4′-azobis(4-cyanovaleric acid) or ACVA, 2,2′-azobis(2,4-dimethylvaleronitrile), cumene hydroperoxide, di-tert-butyl peroxide or tert-butyl hydroperoxide.

The process for preparing the thickening composition (C) will preferably comprise one or more of the following features:
the process comprises a step of removing at least some of the oil (H1) included in composition (C). This allows more concentrated compositions (C) to be obtained,
polymerization is performed at a temperature of between 40° C. and 80° C.,
a crosslinking monomer (AR) is added in step b),
the crosslinking monomer is a diethylenic or polyethylenic crosslinking monomer notably chosen from ethylene glycol dimethacrylate, diethylene glycol diacrylate, ethylene glycol diacrylate, diallylurea, triallylamine, trimethylolpropane triacrylate and methylenebis(acrylamide),
step a) of producing a composition (C0) of itaconic acid mono- and/or oligoesters comprises the following substeps:
i) introduction into a reactor of a biobased alcohol or polyol selected from the elements of the group consisting of compounds in liquid form:
   oligoglycerols having the following structure:

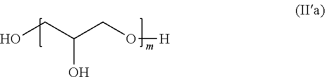

with m representing an integer greater than or equal to 2 and less than or equal to 10,
   natural polyols having the following structure:

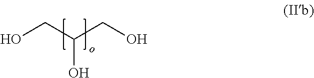

with o representing a number equal to 2 (erythritol), or equal to 3 (xylitol), or equal to 4 (sorbitol),
   dehydrated natural polyols having the following structure:

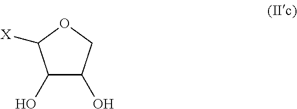

with X representing a hydrogen atom (erythritan), or the radical —CH2-OH (xylitan) or the radical —CH(OH)—CH2-OH (sorbitan),
   aliphatic fatty alcohols having the following structure:

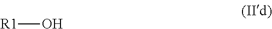

with R1 representing a linear or branched, saturated or unsaturated, hydrocarbon-based aliphatic radical including from 6 to 36 carbon atoms and optionally substituted with one or more hydroxyl groups.

According to a particular aspect, R1 represents a hydrocarbon-based aliphatic radical chosen from n-hexyl, n-heptyl, n-octyl, n-decyl, n-dodecyl, n-tetradecyl, n-hexadecyl, n-octadecyl, oleyl, linoleyl, linolenyl, arachidyl, behenyl, erucyl and 12-hydroxystearyl radicals,
the α,ω-diols of formula (II′e) below:

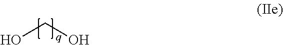

with q representing an integer greater than or equal to 1 and less than or equal to 12,
   the compounds of formula (II′f) below:

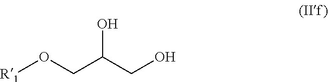

with R′₁ representing a linear or branched, saturated or unsaturated, hydrocarbon-based aliphatic radical including from 6 to 36 carbon atoms and optionally substituted with one or more hydroxyl groups.

According to a particular aspect, R′₁ represents a hydrocarbon-based aliphatic radical chosen from n-hexyl, n-heptyl, n-octyl, n-decyl, n-dodecyl, n-tetradecyl, n-hexadecyl, n-octadecyl, oleyl, linoleyl, linolenyl, arachidyl, behenyl, erucyl and 12-hydroxystearyl radicals. More particularly, the compound of formula (II′f) is batyl alcohol, the structure of which is as follows:

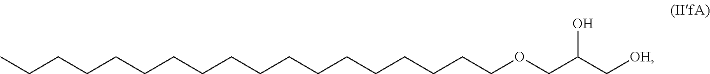 [SMILES: CCCCCCCCCCCCCCCCCCOCC(O)CO]

the compounds of formula (II′g) below:

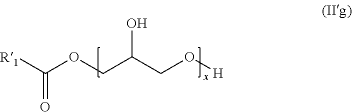

with R′₁—C(═O) representing a linear or branched, saturated or unsaturated, hydrocarbon-based acyl radical including from 12 to 24 carbon atoms, and more particularly a radical chosen from the elements of the group consisting of dodecanoyl, tetradecanoyl, hexadecanoyl, octadecanoyl, eicosanoyl, docosanoyl, oleyl, linoleyl, linolenoyl or isostearyl radicals, and with x representing a natural integer greater than or equal to 1 and less than or equal to 6.

More particularly, the compound of formula (II′g) is glyceryl monostearate having the following formula;

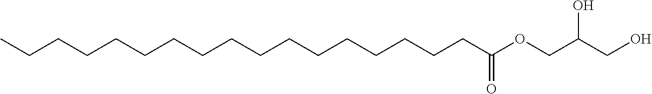 [SMILES: CCCCCCCCCCCCCCCCCC(=O)OCC(O)CO]

(II′gA). More particularly, the compound of formula (II′g) is polyglyceryl-6 laurate, the structure of which is as follows:

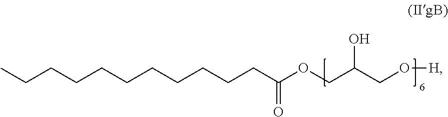

ii) itaconic acid (I), a polymerization inhibitor and then an esterification catalyst are added to the reactor under mechanical stirring,
iii) the medium in the reactor is maintained at a temperature of between 120° C. and 180° C., under partial pressure, to perform an esterification reaction, and
iv) a composition of itaconic acid mono- and/or oligoesters is recovered.

For the purposes of the present invention, the term “polymerization inhibitor” denotes compounds which, when added to monomers, regulate or rapidly stop the undesired initiation of polymerization of said monomers; among these polymerization inhibitors, mention may be made of quinones, hydroquinone, methylhydroquinone, 1,4-benzoquinone, 2-tert-butyl-1,4-benzoquinone, catechols, for instance 4-tert-butylpyrocatechol, 2,6-di-tert-butylphenol, 6-tert-butyl-2,4-xylenol, tert-butylhydroquinone, 4-methoxyphenol, 2,6-di-tert-butyl-p-cresol, aromatic nitro compounds such as nitrobenzenes, nitrophenols, nitrocresols, nitroso compounds, phenothiazine, 1,1-diphenyl-2-picrylhydrazyl, and metal salts such as copper salts, for example copper(II) dibutyldithiocarbamate,
substeps i) to iii) are performed at a temperature of between 12° and 180° C.,
substep iii) is performed at a pressure of between 500 and 20 mbar and, more particularly, between 500 and 200 mbar,
substep iii) lasts between 30 minutes and 10 hours,
in substep ii), the polymerization inhibitor is chosen from 1,4-benzoquinone, 2-tert-butyl-1,4-benzoquinone, phenothiazine, 6-tert-butyl-2,4-xylenol, copper(II) dibutyldithiocarbamate, 1,1-diphenyl-2-picrylhydrazyl, 2,6-di-tert-butyl-p-cresol, 4-tert-butylpyrocatechol, 2,6-di-tert-butylphenol, hydroquinone, tert-butylhydroquinone and 4-methoxyphenol,
in substep ii), the esterification catalyst is an acid chosen from sulfuric, hydrochloric, phosphoric, nitric, hypophosphorous, methanesulfonic, para-toluenesulfonic and trifluoromethanesulfonic acids and acidic ion-exchange resins.

It should be noted that, when a fatty alcohol of formula (IId) is used, a composition (AIId) containing a monoester of formula (AIId1) and/or a diester of formula (AIId2) is obtained.

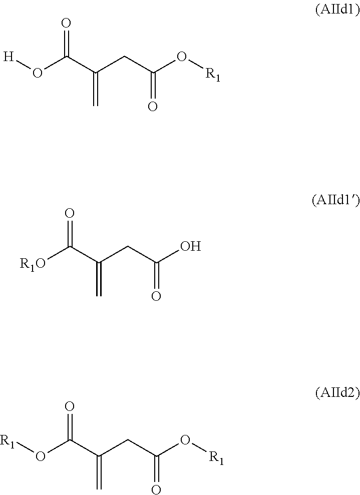

Likewise, when an α,ω-diol of formula (IIe) is used, a composition (AIIe) containing a monoester of formula (IIe1) and/or diesters of formulae (IIe1′) or (IIe2) comprising a single ethylenic bond and/or a diester of formula (IIe3) comprising two ethylenic bonds and/or a tetraester of formula (IIe4) comprising three ethylenic bonds is obtained.

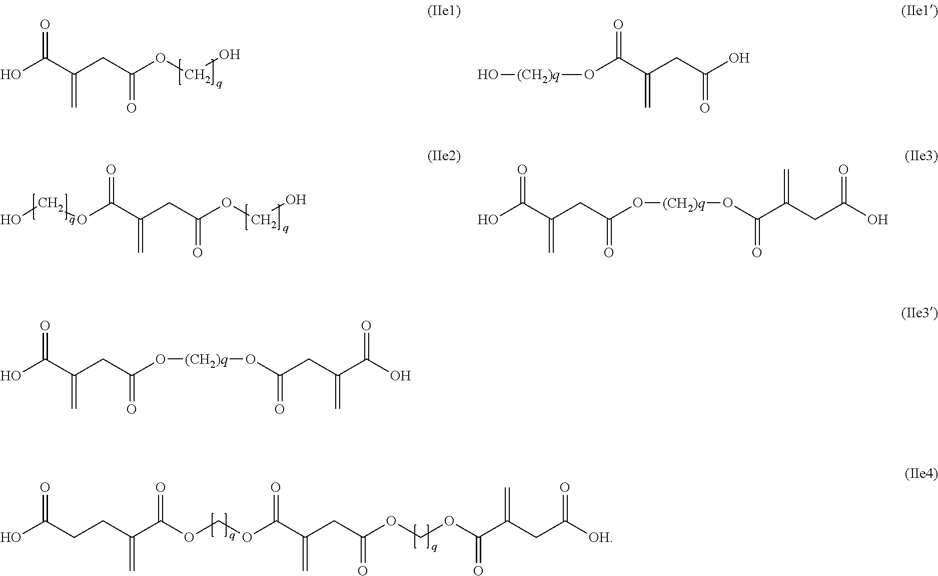

The stoichiometric mole ratio (II′a)/(I) is greater than or equal to 0.25 and less than or equal to 2.0.

The stoichiometric mole ratio (II′b)/(I) is greater than or equal to 0.25 and less than or equal to 2.0.

The stoichiometric mole ratio (II′c)/(I) is greater than or equal to 0.25 and less than or equal to 2.0.

The stoichiometric mole ratio (II′d)/(I) is greater than or equal to 0.25 and less than or equal to 2.0.

The stoichiometric mole ratio (II′e)/(I) is greater than or equal to 0.25 and less than or equal to 2.0.

The stoichiometric mole ratio (II′f)/(I) is greater than or equal to 0.25 and less than or equal to 2.0.

The stoichiometric mole ratio (II′g)/(I) is greater than or equal to 0.25 and less than or equal to 2.0.

The compositions of itaconic acid mono- or oligoesters (A) are characterized by measurements of macroscopic indices (acid and ester numbers) and by their residual itaconic acid content measured by high-performance liquid chromatography (HPLC) coupled to an ultraviolet (UV) detector with internal calibration.

Finally, a degree of itaconic acid conversion is determined. A degree of conversion of greater than or equal to 75% must be achieved in order for the esterification reactions to be considered as having proceeded correctly.

Examples of foaming and/or detergent surfactants that may be combined with said thickening composition (C) as defined previously in said composition (F) include anionic, cationic, amphoteric or nonionic foaming and/or detergent surfactants.

The foaming and/or detergent anionic surfactants that may be combined with said thickening composition (C) as defined previously in said composition (F) include alkali metals salts, alkaline-earth metal salts, ammonium salts, amine salts or amino alcohol salts of alkyl ether sulfates, of alkyl sulfates, of alkylamido ether sulfates, of alkylaryl polyether sulfates, of monoglyceride sulfates, of α-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkylsulfoacetates, of alkyl sarcosinates, of acylisethionates, of N-acyl taurates, of acyl lactylates, of N-acylamino acid derivatives, of N-acyl peptide derivatives, of N-acyl protein derivatives, of N-acyl fatty acid derivatives. The foaming and/or detergent amphoteric surfactants that may be combined with said thickening composition (C) as defined previously in said composition (F) include alkyl betaines, alkylamido betaines, sultaines, alkylamidoalkyl sulfobetaines, imidazoline derivatives, phosphobetaines, amphopolyacetates and amphopropionates. The foaming and/or detergent cationic surfactants that may be combined with said thickening composition (C) as defined previously in said composition (F) particularly include quaternary ammonium derivatives.

The foaming and/or detergent nonionic surfactants that may be combined with said thickening composition (C) as defined previously in said composition (F) more particularly include alkyl polyglycosides including a linear or branched and saturated or unsaturated aliphatic radical including from 8 to 16 carbon atoms, such as octyl polyglucoside, decyl polyglucoside, undecylenyl polyglucoside, dodecyl polyglucoside, tetradecyl polyglucoside, hexadecyl polyglucoside or 1,12-dodecanediyl polyglucoside; ethoxylated hydrogenated castor oil derivatives, such as the product sold under the INCI name PEG-40 hydrogenated castor oil; polysorbates, such as Polysorbate 20, Polysorbate 40, Polysorbate 60, Polysorbate 70, Polysorbate 80 or Polysorbate 85; coconut kernel amides; or N-alkylamines.

Examples of thickening and/or gelling surfactants that may be combined with said thickening composition (C) as defined previously in said composition (F) include optionally alkoxylated alkyl polyglycoside fatty esters, such as ethoxylated methyl polyglucoside esters, for example the PEG 120 methyl glucose trioleate and the PEG 120 methyl glucose dioleate sold respectively under the names Glucamate™ LT and Glucamate™ DOE-120; alkoxylated fatty esters, such as the PEG 150 pentaerythrityl tetrastearate sold under the name Crothix™ DS53 or the PEG 55 propylene glycol oleate sold under the name Antil™ 141; fatty-chain polyalkylene glycol carbamates, such as the PPG-14 laureth isophoryl dicarbamate sold under the name Elfacos™ T211 or the PPG-14 palmeth-60 hexyl dicarbamate sold under the name Elfacos™ GT2125.

Examples of thickeners and/or gelling agents that may be combined with said thickening composition (C) as defined previously in said composition (F) include polysaccharides consisting only of monosaccharides, such as glucans or glucose homopolymers, glucomannoglucans, xyloglycans, galactomannans in which the degree of substitution (DS) of the D-galactose units on the main D-mannose chain is between 0 and 1, and more particularly between 1 and 0.25, such as galactomannans originating from cassia gum (DS=1/5), from locust bean gum (DS=1/4), from tara gum (DS=1/3), from guar gum (DS=1/2) or from fenugreek gum (DS=1).

Examples of thickening and/or gelling agents that may be combined with said thickening composition (C) as defined previously in said composition (F) include polysaccharides consisting of monosaccharide derivatives, such as sulfated galactans and more particularly carrageenans and agar, uronans and more particularly algins, alginates and pectins, heteropolymers of monosaccharides and of uronic acids and more particularly xanthan gum, gellan gum, gum arabic exudates and karaya gum exudates, or glucosaminoglycans.

Examples of thickening and/or gelling agents that may be combined with said thickening composition (C) as defined previously in said composition (F) include cellulose, cellulose derivatives, such as methyl cellulose, ethyl cellulose or hydroxypropyl cellulose, silicates, starch, hydrophilic starch derivatives or polyurethanes.

Examples of stabilizers that may be combined with said thickening composition (C) as defined previously in said composition (F) include monocrystalline waxes and more particularly ozokerite, mineral salts, such as sodium chloride or magnesium chloride, or silicone polymers, such as polysiloxane polyalkyl polyether copolymers.

Examples of solvents that may be combined with said thickening composition (C) as defined previously in said composition (F) include water, organic solvents, such as glycerol, diglycerol, glycerol oligomers, ethylene glycol, propylene glycol, butylene glycol, 1,3-propanediol, 1,2-propanediol, hexylene glycol, diethylene glycol, xylitol, erythritol, sorbitol, water-soluble alcohols, such as ethanol, isopropanol or butanol, or mixtures of water and of said organic solvents.

Examples of thermal or mineral waters that may be combined with said thickening composition (C) as defined previously in said composition (F) include thermal or mineral waters having a mineralization of at least 300 mg/I, in particular Avène water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

Examples of hydrotropic agents that may be combined with said thickening composition (C) as defined previously in said composition (F) include xylenesulfonates, cumenesulfonates, hexyl polyglucoside, 2-ethylhexyl polyglucoside and n-heptyl polyglucoside.

Examples of emulsifying surfactants that may be combined with said thickening composition (C) as defined previously in said composition (F) include nonionic surfactants, anionic surfactants or cationic surfactants.

Examples of emulsifying nonionic surfactants that may be combined with said thickening composition (C) as defined previously in said composition (F) include fatty acid esters of sorbitol, for instance the products sold under the names Montane™ 40, Montane™ 60, Montane™ 70, Montane™ 80 and Montane™ 85; compositions comprising glyceryl stearate and stearic acid ethoxylated with between 5 mol and 150 mol of ethylene oxide, for instance the composition comprising stearic acid ethoxylated with 135 mol of ethylene oxide and glyceryl stearate sold under the name Simulsol™ 165; mannitan esters, ethoxylated mannitan esters; sucrose esters; methyl glucoside esters; alkyl polyglycosides including a linear or branched, saturated or unsaturated aliphatic radical, and including from 14 to 36 carbon atoms, for instance tetradecyl polyglucoside, hexyldecyl polyglucoside, octadecyl polyglucoside, hexadecyl polyxyloside, octadecyl polyxyloside, eicosyl polyglucoside, dodecosyl polyglucoside, 2-octyldodecyl polyxyloside, 12-hydroxystearyl polyglucoside; compositions of linear or branched, saturated or unsaturated fatty alcohols including from 14 to 36 carbon atoms and of alkyl polyglycosides as described previously, for example the compositions sold under the names Montanov™ 68, Montanov™ 14, Montanov™ 82, Montanov™ 202, Montanov™ S, Montanov™ W018, Montanov™ L, Fluidanov™ 20X and Easynov™

Examples of anionic surfactants that may be combined with said thickening composition (C) as defined previously in said composition (F) include glyceryl stearate citrate, cetearyl sulfate, soaps, such as sodium stearate or triethanolammonium stearate, and N-acylamino acid derivatives which are salified, for example stearoyl glutamate.

Examples of cationic emulsifying surfactants that may be be combined with said thickening composition (C) as defined previously in said composition (F) include amine oxides, quaternium-82 and the surfactants described in patent application WO 96/00719 and mainly those whose fatty chain comprises at least 16 carbon atoms.

Examples of opacifying and/or nacreous agents that may be combined with said thickening composition (C) as defined previously in said composition (F) include sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate or fatty alcohols including from 12 to 22 carbon atoms.

Examples of texturing agents that may be combined with said thickening composition (C) as defined previously in said composition (F) include N-acylamino acid derivatives, such as lauroyl lysine sold under the name Aminohope™ LL, starch octenylsuccinate sold under the name Dryflo™, myristyl polyglucoside sold under the name Montanov™ 14, cellulose fibers, cotton fibers, chitosan fibers, talc, sericite or mica. Examples of deodorant agents that may be combined with said thickening composition (C) as defined previously in said composition (F) include alkali metal silicates, zinc salts, such as zinc sulfate, zinc gluconate, zinc chloride or zinc lactate; quaternary ammonium salts, such as cetyltrimethylammonium salts or cetylpyridinium salts; glycerol derivatives, such as glyceryl caprate, glyceryl caprylate or polyglyceryl caprate; 1,2-decanediol, 1,3-propanediol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, such as the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, or the complex of aluminum sesquichlorohydrate and of polyethylene glycol.

Examples of waxes that may be combined with said thickening composition (C) as defined previously in said composition (F) include beeswax, carnauba wax, candelilla wax, ouricury wax, Japan wax, cork fiber wax, sugarcane wax, paraffin waxes, lignite waxes, microcrystalline waxes, lanolin wax; ozokerite, polyethylene wax, silicone waxes, plant waxes, fatty alcohols and fatty acids that are solid at room temperature, or glycerides that are solid at room temperature. In the present patent application, the term “waxes” refers to compounds and/or mixtures of compounds which are water-insoluble, and which have a solid appearance at a temperature of greater than or equal to 45° C.

Examples of active principles that may be combined with said thickening composition (C) as defined previously in said composition (F) include vitamins and their derivatives, notably their esters, such as retinol (vitamin A) and its esters (for example retinyl palmitate), ascorbic acid (vitamin C) and its esters, sugar derivatives of ascorbic acid (such as ascorbyl glucoside), tocopherol (vitamin E) and its esters (such as tocopheryl acetate), vitamin B3 or B10 (niacinamide and its derivatives); compounds showing a lightening or depigmenting action on the skin, such as w-undecylenoyl phenylalanine sold under the name Sepiwhite™ MSH, Sepicalm™ VG, the glycerol monoester and/or diester of w-undecylenoyl phenylalanine, w-undecylenoyl dipeptides, arbutin, kojic acid, hydroquinone; compounds showing a calmative action, notably Sepicalm™ S, allantoin and bisabolol; anti-inflammatory agents; compounds showing a moisturizing action, such as urea, hydroxyureas, glycerol, polyglycerols, glyceryl glucoside, diglyceryl glucoside, polyglyceryl glucosides, xylityl glucoside; polyphenol-rich plant extracts, such as grape extracts, pine extracts, wine extracts or olive extracts; compounds showing a slimming or lipolytic action, such as caffeine or its derivatives, Adiposlim™, Adipoless™, fucoxanthin; N-acyl proteins; N-acyl peptides, such as Matrixyl™; N-acyl amino acids; partial hydrolyzates of N-acyl proteins; amino acids; peptides; total hydrolyzates of proteins; soybean extracts, for example Raffermine™; wheat extracts, for example Tensine™ or Gliadine™; plant extracts, such as tannin-rich plant extracts, isoflavone-rich plant extracts or terpene-rich plant extracts; extracts of freshwater or marine algae; marine plant extracts; marine extracts in general, such as corals; essential waxes; bacterial extracts; ceramides; phospholipids; compounds showing an antimicrobial action or a purifying action, such as Lipacide™ C8G, Lipacide™ UG, Sepicontrol™ A5, Octopirox™ or Sensiva™ SC50; compounds showing an energizing or stimulating property, such as Physiogenyl™, panthenol and its derivatives, such as Sepicap™ MP; antiaging active agents, such as Sepilift™ DPHP, Lipacide™ PVB, Sepivinol™, Sepivital™, Manoliva™, Phyto-Age™, Timecode™; Survicode™; antiphotoaging active agents; active agents which protect the integrity of the dermoepidermal junction; active agents which increase the synthesis of the components of the extracellular matrix, such as collagen, elastins or glycosaminoglycans; active agents which act favorably on chemical cell communication, such as cytokines, or physical cell communication, such as integrins; active agents which create a sensation of “heating” on the skin, such as activators of cutaneous microcirculation (such as nicotinic acid derivatives) or products which create a sensation of “freshness” on the skin (such as menthol and derivatives); active agents which improve cutaneous microcirculation, for example venotonics; draining active agents; active agents having a decongestant purpose, such as Ginkgo biloba, ivy, horse chestnut, bamboo, Ruscus, butcher's broom, Centella asiatica, fucus, rosemary or willow extracts; agents for tanning or browning the skin, for example dihydroxyacetone (DHA), erythrulose, mesotartaric aldehyde, glutaraldehyde, glyceraldehyde, alloxan or ninhydrin, plant extracts, for example extracts of red woods of the genus Pterocarpus and of the genus Baphia, such as Pteropcarpus santalinus, Pterocarpus osun, Pterocarpus soyauxii, Pterocarpus erinaceus, Pterocarpus indicus or Baphia nitida, such as those described in the European patent application EP 0 971 683; agents known for their action in facilitating and/or accelerating tanning and/or browning of human skin, and/or for their action in coloring human skin, for example carotenoids (and more particularly β-carotene and γ-carotene), the product sold under the brand name Carrot Oil (INCI name: Daucus carrota, Helianthus annuus sunflower oil) by the company Provital, which contain carotenoids, vitamin E and vitamin K; tyrosine and/or its derivatives, known for their effect on accelerating the tanning of human skin in combination with exposure to ultraviolet radiation, for example the product sold under the brand name SunTan Accelerator™ by the company Provital, which contains tyrosine and riboflavins (vitamin B), the complex of tyrosine and of tyrosinase sold under the brand name Zymo Tan Complex by the company Zymo Line, the product sold under the brand name MelanoBronze™ (INCI name: Acetyl Tyrosine, Monk's pepper extract (Vitex agnus-castus)) by the company Mibelle, which contains acetyl tyrosine, the product sold under the brand name Unipertan VEG-24/242/2002 (INCI name: Butylene Glycol and Acetyl Tyrosine and Hydrolyzed Vegetable Protein and Adenosine Triphosphate) by the company Unipex, the product sold under the brand name Try-Excell™ (INCI name: Oleoyl tyrosine and Luffa cylindrica (seed) oil and oleic acid) by the company Sederma, which contains extracts of marrow seeds (or loofah oil), the product sold under the brand name Actibronze™ (INCI name: Hydrolyzed wheat protein and acetyl tyrosine and copper gluconate) by the company Alban Muller, the product sold under the brand name Tyrostan™ (INCI name: Potassium caproyl tyrosine) by the company Synerga, the product sold under the brand name Tyrosinol (INCI name: Sorbitan Isostearate, Glyceryl Oleate, Caproyl Tyrosine) by the company Synerga, the product sold under the brand name InstaBronze™ (INCI name: Dihydroxyacetone and Acetyl Tyrosine and Copper Gluconate) by the company Alban Muller, the product sold under the brand name Tyrosilane (INCI name: Methylsilanol and Acetyl Tyrosine) by the company Exymol; peptides known for their effect in activating melanogenesis, for example the product sold under the brand name Bronzing SF Peptide Powder (INCI name: Dextran and Octapeptide-5) by the company Infinitec Activos, the product sold under the brand name Melitane (INCI name: Glycerin and Aqua and Dextran and Acetyl Hexapeptide-1) comprising acetyl hexapeptide-1 known for its α-MSH agonist action, the product sold under the brand name Melatimes Solutions™ (INCI name: Butylene glycol, Palmitoyl Tripeptide-40) by the company Lipotec, sugars and sugar derivatives, for example the product sold under the brand name Tanositol™ (INCI name: inositol) by the company Provital, the product sold under the brand name Thalitan™ (or Phycosaccharide™ AG) by the company CODIF International (INCI name: Aqua and Hydrolyzed algin (Laminaria digitata) and magnesium sulfate and manganese sulfate) containing an oligosaccharide of marine origin (guluronic acid and mannuronic acid chelated with magnesium and manganese ions), the product sold under the brand name Melactiva™ (INCI name: Maltodextrin, Mucuna pruriens Seed Extract) by the company Alban Muller, compounds rich in flavonoids, for example the product sold under the brand name Biotanning (INCI name: Hydrolyzed citrus Aurantium dulcis fruit extract) by the company Silab and known to be rich in lemon flavonoids (of the hesperidin type); agents intended for treating head hair and/or bodily hair, for example agents for protecting the melanocytes of the hair follicle, intended to protect said melanocytes against cytotoxic agents responsible for the senescence and/or apoptosis of said melanocytes, such as mimetics of DOPAchrome tautomerase activity, chosen from those described in the European patent application published under the number EP 1 515 688 A2, synthetic SOD mimetic molecules, for example manganese complexes, antioxidant compounds, for example cyclodextrin derivatives, siliceous compounds derived from ascorbic acid, lysine or arginine pyrrolidone carboxylate, combinations of mono- and diesters of cinnamic acid and of vitamin C, and more generally those mentioned in the European patent application published under the number EP 1 515 688 A2.

Examples of antioxidants that may be combined with said thickening composition (C) as defined previously in said composition (F) include EDTA and its salts, citric acid, tartaric acid, oxalic acid, BHA (butylated hydroxyanisole), BHT (butylated hydroxytoluene), tocopherol derivatives, such as tocopheryl acetate, mixtures of antioxidant compounds, such as Dissolvine GL 47S sold by the company AkzoNobel under the INCI name: Tetrasodium Glutamate Diacetate.

Examples of sunscreens that may be combined with said thickening composition (C) as defined previously in said composition (F) include all those appearing in the amended Cosmetics Directive 76/768/EEC, Annex VII.

The organic sunscreens that may be combined with said thickening composition (C) as defined previously in said composition (F) include the family of benzoic acid derivatives, for instance para-aminobenzoic acids (PABA), notably monoglyceryl esters of PABA, ethyl esters of N,N25-propoxy PABA, ethyl esters of N,N-diethoxy PABA, ethyl esters of N,N-dimethyl PABA, methyl esters of N,N-dimethyl PABA and butyl esters of N,N-dimethyl PABA; the family of anthranilic acid derivatives, for instance homomenthyl-N-acetyl anthranilate; the family of salicylic acid derivatives, for instance amyl salicylate, homomenthyl salicylate, ethylhexyl salicylate, phenyl salicylate, benzyl salicylate and p-isopropanolphenyl salicylate; the family of cinnamic acid derivatives, for instance ethylhexyl cinnamate, ethyl 4-isopropylcinnamate, methyl 2,5-diisopropylcinnamate, p-methoxypropyl cinnamate, p-methoxyisopropyl cinnamate, p-methoxyisoamyl cinnamate, p-methoxyoctyl cinnamate (p-methoxy-2-ethylhexyl cinnamate), p-methoxy-2-ethoxyethyl cinnamate, p-methoxycyclohexyl cinnamate, ethyl-α-cyano-β-phenyl cinnamate, 2-ethylhexyl-α-cyano-β-phenyl cinnamate or glyceryl di-para-methoxymono-2-ethylhexanoyl cinnamate; the family of benzophenone derivatives, for instance 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl 4′-phenylbenzophenone-2-5-carboxylate, 2-hydroxy-4-n-octyloxybenzophenone, 4-hydroxy-3-carboxybenzophenone; 3-(4′-methylbenzylidene)-d,l-camphor, 3-(benzylidene)-d,l-camphor, camphor benzalkonium methosulfate; urocanic acid, ethyl urocanate; the family of sulfonic acid derivatives, for instance 2-phenylbenzimidazole-5-sulfonic acid and salts thereof; the family of triazine derivatives, for instance hydroxyphenyltriazine, ethylhexyloxyhydroxyphenyl-4-methoxyphenyltriazine, 2,4,6-trianillino(p-carbo-2′-ethylhexyl-1′-oxy)-1,3,5-triazine, 4,4-((6-(((1,1-dimethylethyl)amino)carbonyl)phenyl)amino)-1,3,5-triazine-2,4-diyldiimino)bis(2-ethylhexyl)benzoate, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; the family of diphenylacrylate derivatives, for instance 2-ethylhexyl 2-cyano-3,3-diphenyl-2-propenoate, ethyl 2-cyano-3,3-diphenyl-2-propenoate; the family of polysiloxanes, for instance benzylidene siloxane malonate.

The mineral sunscreens, also known as “mineral sunblocks”, that may be combined with said thickening composition (C) as defined previously in said composition (F) include titanium oxides, zinc oxides, cerium oxide, zirconium oxide, yellow, red or black iron oxides, or chromium oxides. These mineral sunblocks may or may not be micronized, may or may not have been subjected to surface treatments and may optionally be in the form of aqueous or oily predispersions.

### DETAILED DESCRIPTION

The invention will now be described in greater detail by means of the examples below.

Examples according to the invention:

### E.1—Preparation of Compositions of Itaconic Acid Mono- and/or Oligoesters (A) According To the Invention

### E.1.1—Preparation of the Glyceryl Monostearate Itaconate Composition (AIIg)

108.5 g (1 molar equivalent) of glyceryl monostearate (II′gA), 39.6 g (1 molar equivalent) of itaconic acid (I) and 1000 ppm (/reaction medium) of 4-methoxyphenol (or MEHQ) are introduced into a reactor at 135° C. with mechanical stirring. 0.5% (/reaction medium) of a 50% aqueous solution of H₃PO₂ is then added and the reaction medium is placed under controlled vacuum at about 500 mbar. The esterification reaction begins (distillation of the water formed) and is continued for 6 hours. After 6 hours, 125.7 g of glyceryl monostearate itaconate composition (AIIg) are isolated and characterized as such without any further steps.

### E.1.2—Preparation of the Batyl Alcohol Itaconate Composition (AIIf)

The same procedure as described in paragraph E.1.1—was performed, replacing the 108.5 g (1 molar equivalent) of glyceryl monostearate (II′gA) and 39.6 g (1 molar equivalent) of itaconic acid (I) with 50.3 g (1 molar equivalent) of batyl alcohol (II′f) and 19.2 g (1 molar equivalent) of itaconic acid (I). 61.2 g of batyl alcohol itaconate composition (AIIf) are isolated and characterized.

### E.1.3—Preparation of the Stearyl Alcohol Itaconate Composition (AIId17)

The same procedure as described in paragraph E.1.1—was performed, replacing the 108.5 g (1 molar equivalent) of glyceryl monostearate (II′gA) and 39.6 g (1 molar equivalent) of itaconic acid (I) with 74.0 g (1 molar equivalent) of stearyl alcohol (II′d17) and 35.9 g (1 molar equivalent) of itaconic acid (I). 100.3 g of stearyl alcohol itaconate composition (AIId17) are isolated and characterized.

### E.1.4—Preparation of the Polyglycerol-6 Laurate Itaconate Composition (AIIh)

The same procedure as described in paragraph E.1.1—was performed, replacing the 108.5 g (1 molar equivalent) of glyceryl monostearate (II′g) and 39.6 g (1 molar equivalent) of itaconic acid (I) with 160.1 g (1 molar equivalent) of polyglycerol-6 laurate (II′gB) and 32.3 g (1 molar equivalent) of itaconic acid (I). 102.4 g of the polyglyceryl-6 laurate itaconate composition (AIIh) are isolated and characterized.

### E.1.5—Results and Conclusions

The analytical features of all the compositions (A) prepared are collated in table 1 below.

TABLE 1

| Reference | | | | | |
|---|---|---|---|---|---|
| Composition | (II′) (mol. eq/ | Acid Number | ESTER Number (mg | (I) residual | Conversion (I) |
| (A) | (I)) | (mg KOH/g) | KOH/g) | (%)^{a)} | (%)^{b)} |
| 5211 JG | (II′a; m = 2) | Not measured | Not measured | 11.1 | 86.4 |
| | (0.33) | | | | |
| 5212 JG | (II′a; m = 2) | 345.1 | 347.8 | 21.1 | 76.6 |
| | (0.25) | | | | |
| 5298 JG | (II′a; m = 6) | 156.1 | 161.0 | Not measured | Not measured |
| | (0.5) | | | | |
| 5375 JG | (II′b; o = 2) | Not measured | Not measured | 12.0 | 84.8 |
| | (0.5) | | | | |
| 5358 JG | (II′b; o = 3) | 285.2 | 315.8 | 14.6 | 79.9 |
| | (0.5) | | | | |
| 5350 JG | (II′b; o = 4) | 265.3 | 295.6 | 12.5 | 82.2 |
| | (0.5) | | | | |
| 5360 JG | (II′c; X = | 344.7 | 296.5 | 6.7 | 91.0 |
| | —CH(OH)—CH₂—OH) | | | | |
| | (0.5) | | | | |
| 5228 JG | (II′d; p = 6) | 208.9 | 256.3 | 6.7 | 89.0 |
| | (1.0) | | | | |
| (AIId17) | (II′d; p = 17) | 77.9 | 150.8 | 3.6 | 89.9 |
| | (1.0) | | | | |
| 5394 JG | (II′d; p = 17) | 61.1 | 102.5 | 0.2 | 99.0 |
| | (2.0) | | | | |
| 5370 JG | (II′e; q = 8) | 302.3 | 292.2 | 10.5 | Not measured |
| | (0.5) | | | | |
| 5361 JG | (II′e; q = 10) | 275.7 | 283.5 | 8.1 | 88.0 |
| | (0.5) | | | | |
| 5302 JG | (II′e; q = 12) | 264.0 | 216.7 | 9.2 | 85.3 |
| | (0.5) | | | | |
| (AIIf) | (II′f) (1.0) | 115.2 | 120.9 | 6.5 | 79.3 |
| (AIIg) | (II′g) (1.0) | 104.3 | 250.3 | 4.7 | 85.0 |
| (AIIh) | (II′gB) (1.0) | 74.0 | 124.3 | 4.0 | Not measured |

^{a)}Determined by HPLC/UV in the presence of an internal standard,
^{b)}Degree of conversion = [1 − ((mass composition (A) × % (I) residual)/mass itaconic acid at start)] × 100.

Table 1—^{Characteristics} of the itaconic acid mono- and/or oligoester compositions (A) prepared.

Overall, the esterification conditions described previously allow the isolation of compositions (A) having ester numbers of between 100 and 350 mg KOH/g, proving the significant formation of esterified species.

In all cases, the acid numbers are greater than 0, ranging between 60 and 350 mg KOH/g. These values indicate that not all the carboxylic acid functions of itaconic acid (I) are esterified.

Finally, the ^{degrees} of conversion calculated from the final mass of composition (A) collected, the percentage of residual itaconic acid (I) contained in this final mass and determined by GC, and also the mass of itaconic acid (I) used initially, are systematically greater than 75%, indicating that the esterification reactions have proceeded satisfactorily.

### E.2—Preparation of the Poly(Itaconates) (P) According to the Invention

### E.2.1—Polymerization of the Glyceryl Monostearate Itaconate Composition (AIIg) in Isopar™ to Obtain Composition (C1)

26.7 g of composition (AIIg) are dissolved in 28.1 g of Isopar™ M in a reactor. The solution is homogenized with stirring at about 400 rpm at a temperature of 50° C., then sparged with nitrogen for 45 min so as to remove oxygen from the medium. The medium is then heated to a temperature of 80° C. 110 mg of lauroyl peroxide are then added to initiate the polymerization reaction. The medium becomes more viscous. After 2 h, 55 mg of lauryl peroxide are then introduced. After maintenance for a further 3 h, composition (C1) takes on a wax-like appearance and is conditioned while hot.

### E.2.2—Polymerization of the Referenced Glyceryl Monostearate Itaconate Composition (AIIg) in a Coco-Caprylate/Caprate Composition Sold Under the Trade Name DUB 810 C) To Obtain Composition (C2)

The same procedure as described in paragraph E.2.1—was performed, replacing the 28.1 g of Isopar™ M oil with 28.1 g of DUB 810 C. Composition (C2) was isolated.

### E.2.3—Polymerization of the Batyl Alcohol Itaconate Composition (AIIf) in DUB 810 C To Obtain Composition (C3)

The same procedure as described in paragraph E.2.2—was performed, replacing the 26.7 g of composition (AIIg) with 26.7 g of composition (AIIf). Composition (C3) was isolated.

### E.2.4—Polymerization of the Referenced Stearyl Alcohol Itaconate Composition (AIId17) in a Mixture of Alkanes, Isoalkanes and Cycloalkanes Sold Under the Trade Name Emogreen™ L19 to Obtain Composition (C4)

The same procedure as described in paragraph E.2.1—was performed, replacing the 28.1 g of Isopar™ M oil with 28.1 g of Emogreen™ L19, and replacing the 26.7 g of composition (AIIg) with 26.7 g of composition (AIId17). Composition (C4) thus obtained was isolated.

### E.2.5—Polymerization of the Referenced Stearyl Alcohol Itaconate Composition (AIId17) in DUB 810C to Obtain Composition (C5)

The same procedure as described in paragraph E.2.4—was performed, replacing the 28.1 g of Emogreen™ L19 with 28.1 g of DUB 810C. Composition (C5) thus obtained was isolated.

### E.2.6—Summary Table

TABLE 2

| Reference | | | |
|---|---|---|---|
| Composition | Monomer(s) of the Polymer (P) | | Mass Ratio (P)/ |
| (C) | [Mass ratio in the case of copolymer] | Oil (H1) | (H1) |
| (C1) | Composition (AIIg) | Isopar ™ M | 0.49/0.51 |
| | Glyceryl monostearate itaconates | | |
| (C2) | Composition (AIIg) | DUB 810 C | 0.49/0.51 |
| | Glyceryl monostearate itaconates | | |
| (C3) | Composition (AIIf) | DUB 810 C | 0.49/0.51 |
| | Batyl alcohol itaconates | | |
| (C4) | Composition (AIId17) | Emogreen | 0.49/0.51 |
| | Stearyl alcohol itaconates | L19 | |
| (C5) | Composition (AIId17) Stearyl alcohol | DUB 810 C | 0.49/0.51 |
| | itaconates | | |

Table 2—Characteristics of the polymers (P) and of the compositions (C) prepared.

### E.3—Evaluation of the Thickening Properties of the Compositions (C)

At 80° C., 0.7 g of compositions (C1) to (C5) are dissolved in 5 g of two different oils (H2): Primol™ 352 and Emogreen™ L19. The solutions thus obtained are cooled to room temperature and their thickening is assessed visually and/or measured at a temperature of 25° C. using a Brookfield (or BKF) viscometer of the LVT type fitted with a suitable spindle (M) and at a speed of 6 rpm (V6).

The results obtained for the observation at 25° C. are collated in Table 3 below.

TABLE 3

| Reference | | |
|---|---|---|
| Composition (C) | Primol ™ 352 | Emogreen ™ L 19 |
| (C1) | ++ | +++ |
| (C2) | ++ | Not measured |
| C3 | ++ | Not measured |
| (C4) | ++ | ++ |
| (C5) | ++ | ++ |

++: Viscous consistency
+++: Gelled consistency

Table 3—Thickening properties on the oils (H2) of the compositions (C).

The results obtained for the viscosity measurements performed using a BKF viscometer, spindle 3, speed 6 rpm at 25° C. are collated in Table 4 below.

TABLE 4

| Reference | Viscosity 25° C. BKF LVT | | Viscosity 25° C. BKF LVT | |
|---|---|---|---|---|
| Composition | M3V6 | | M3V6 | |
| (C) | Primol ™ 352 | | Emogreen ™ L 19 | |
| (C1) | 1 000 | mPa · s | ≥1 000 | mPa · s |
| (C2) | 1 000 | mPa · s | Not measured | |
| (C3) | 5 100 | mPa · s | 38 350 | mPa · s |
| (C4) | 2 240 | mPa · s | 1 500 | mPa · s |
| (C5) | 1 480 | mPa · s | 800 | mPa · s |

In general, viscous or gelled consistencies were visualized for both oils (H2) differing in chemical structure and polarity.

Thickening is confirmed by measurement of the dynamic viscosities at 25° C., since at this same temperature of 25° C., Primol™ 352 shows a viscosity of 165 mPa·s (BKF LVT M2V6) and Emogreen™ L19 shows a viscosity of less than or equal to 10 mPa·s (BKF LVT M2V6).

None of the thickened oils, namely (C1) to (C5), show any runniness or tack or have a greasy feel on the skin after application to said skin (evaluated by a panel of duly qualified and trained evaluators).

These examples show that the process performed according to the invention advantageously allows a gelling agent to be obtained in liquid form, containing a polymer obtained by polymerization of monomers derived from starting materials of plant origin, which is ready for use immediately on preparation in the oil (H1), unlike the gelling polymers of the prior art which are in powder form.

1. A composition (C) for thickening polar and/or apolar oils (H2), comprising at least one oil (H1) and a poly(itaconate) (P), the poly(itaconate) (P) consisting of:
at least one monomer unit derived from itaconic acid of formula (I) in free acid form or partially or totally salified form: 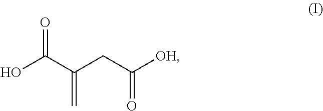
and
at least one monomer unit derived from a compound of formula (II): 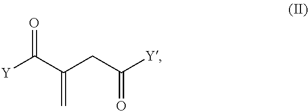
with Y and Y′, which are identical, representing the radical —OR₁, or Y and Y′, which are different, representing either the —OH radical or the radical —OR₁, with R1 representing:
a linear or branched, saturated or unsaturated, aliphatic hydrocarbon-based radical (IIa) including from 6 to 36 carbon atoms and optionally substituted with one or more hydroxyl groups,
or
a radical of formula (IIb) in which R₂ represents a linear or branched, saturated or unsaturated, aliphatic hydrocarbon-based radical including from 6 to 36 carbon atoms and optionally substituted with one or more hydroxyl groups: 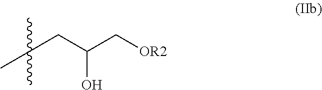
or
a radical of formula (IIc) in which R₃—C(═O) represents a linear or branched, saturated or unsaturated hydrocarbon-based acyl radical containing from 12 to 24 carbon atoms, and more particularly a radical chosen from the elements of the group consisting of dodecanoyl, tetradecanoyl, hexadecanoyl, octadecanoyl, eicosanoyl, docosanoyl, oleyl, linoleyl, linolenoyl or isostearyl radicals, and in which x represents a natural integer greater than or equal to 1 and less than or equal to 6: 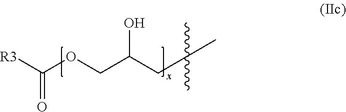
or
a radical of formula (IId) in which m represents an integer greater than or equal to 2 and less than or equal to 10: 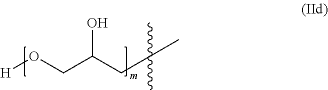
or
a radical of formula (IIe) in which o represents an integer greater than or equal to 2 and less than or equal to 4: 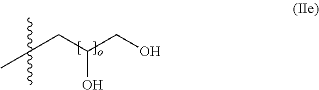
or
a radical of formula (IIf): 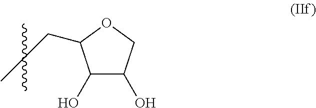
or
a radical of formula (IIg): 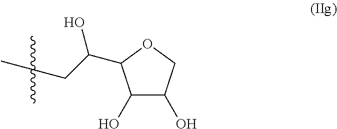
or
a radical of formula (IIh) in which q represents an integer greater than or equal to 1 and less than or equal to 12:

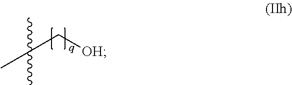

2. The thickening composition (C) as claimed in claim 1, further comprising between 20% and 80% by mass of oil (H1) and between 20% and 80% by mass of poly(itaconate) (P).

3. The thickening composition (C) as claimed in either claim 1, wherein the oil (H1) is an oil which is in liquid form at a temperature of between 4° C. and 45° C.

4. The thickening composition (C) as claimed in claim 1, wherein the oil H1 is a mineral oil or an oil of plant origin, or a mixture of these two types of oil.

5. A topical formulation (F) comprising a composition (C) as defined in claim 1.

6. A process for preparing a thickening composition (C) as defined in claim 1, comprising the following steps:
a) production of a composition (C0) of itaconic acid mono- and/or oligoesters comprising a compound of formula (I) in free acid form or partially or totally salified form: 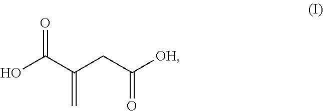
and
a compound of formula (II): 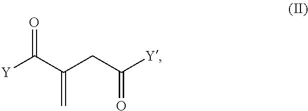
with Y and Y′, which are identical, representing the radical —OR₁, or Y and Y′, which are different, representing either the —OH radical or the radical —OR₁, with R₁ representing:
a linear or branched, saturated or unsaturated, aliphatic hydrocarbon-based radical (IIa) including from 6 to 36 carbon atoms and optionally substituted with one or more hydroxyl groups,
or
a radical of formula (IIb) in which R₂ represents a linear or branched, saturated or unsaturated, aliphatic hydrocarbon-based radical including from 6 to 36 carbon atoms and optionally substituted with one or more hydroxyl groups: 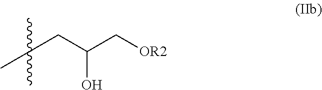
or
a radical of formula (IIc) in which R₃—C(═O) represents a linear or branched, saturated or unsaturated hydrocarbon-based acyl radical containing from 12 to 24 carbon atoms, and more particularly a radical chosen from the elements of the group consisting of dodecanoyl, tetradecanoyl, hexadecanoyl, octadecanoyl, eicosanoyl, docosanoyl, oleyl, linoleyl, linolenoyl or isostearyl radicals, and in which x represents a natural integer greater than or equal to 1 and less than or equal to 6: 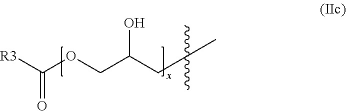
or
a radical of formula (IId) in which m represents an integer greater than or equal to 2 and less than or equal to 10: 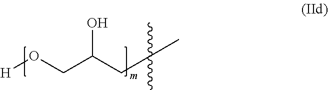
or
a radical of formula (IIe) in which o represents an integer greater than or equal to 2 and less than or equal to 4: 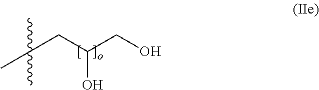
or
a radical of formula (IIf): 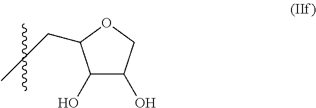
or
a radical of formula (JIg): 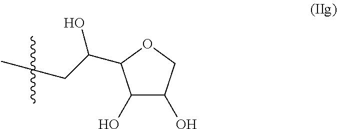
or
a radical of formula (IIh) in which q represents an integer greater than or equal to 1 and less than or equal to 12: 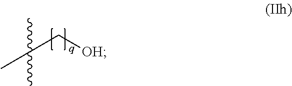
b) preparation of a mixture (M1) of the composition (C0) produced in step a) with an oil (H1),
c) polymerization of the mixture (M1) initiated by introducing a thermal radical initiator into said mixture (M1) obtained on conclusion of step b), and
d) recovery of composition (C).

7. The process as claimed in claim 6, further comprising a step of removing at least some of the oil (H1) included in composition (C).

8. The process as claimed in claim 6, wherein the polymerization is performed at a temperature of between 40° C. and 80° C.

9. The process as claimed in claim 6, wherein, in step b), a crosslinking monomer (AR) is added.

10. The process as claimed in claim 9, wherein the crosslinking monomer (AR) is a diethylenic or polyethylenic crosslinking monomer notably chosen from ethylene glycol dimethacrylate, diethylene glycol diacrylate, ethylene glycol diacrylate, diallylurea, triallylamine, trimethylolpropane triacrylate and methylenebis(acrylamide).

11. The process as claimed in claim 6, wherein step a) of producing a composition (C0) of itaconic acid mono- and/or oligoesters comprises the following substeps:
i) introduction into a reactor of a biobased alcohol or polyol selected from the elements of the group consisting of compounds in liquid form:
oligoglycerols having the following structure: 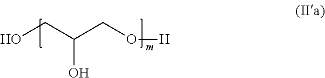
with m representing an integer greater than or equal to 2 and less than or equal to 10,
natural polyols having the following structure: 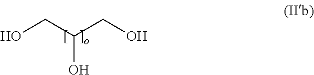
with o representing a number equal to 2 (erythritol), or equal to 3 (xylitol), or equal to 4 (sorbitol),
dehydrated natural polyols having the following structure: 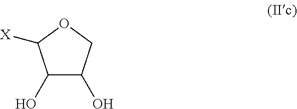
with X representing a hydrogen atom (erythritan), or the radical —CH2-OH (xylitan) or the radical —CH(OH)—CH2-OH (sorbitan),
aliphatic fatty alcohols having the following structure:
R1—OH (II′d)
with R1 representing a linear or branched, saturated or unsaturated, hydrocarbon-based aliphatic radical including from 6 to 36 carbon atoms and optionally substituted with one or more hydroxyl groups,
the α,ω-diols of formula (II′e) below: 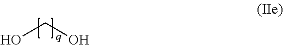
with q representing an integer greater than or equal to 1 and less than or equal to 12,
the compounds of formula (II′f) below: 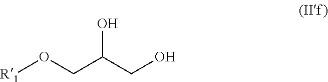
with R′₁ representing a linear or branched, saturated or unsaturated, hydrocarbon-based aliphatic radical including from 6 to 36 carbon atoms and optionally substituted with one or more hydroxyl groups,
the compounds of formula (II′g) below: 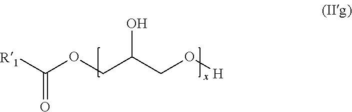
with R′₁—C(═O) representing a linear or branched, saturated or unsaturated hydrocarbon-based acyl radical including from 12 to 24 carbon atoms, and x representing an integer greater than or equal to 1 and less than or equal to 6,
ii) itaconic acid (I), a polymerization inhibitor and then an esterification catalyst are added to the reactor under mechanical stirring,
iii) the medium in the reactor is maintained at a temperature of between 120° C. and 180° C., under partial pressure, to perform an esterification reaction, and
iv) a composition of itaconic acid mono- and/or oligoesters is recovered.

12. The process as claimed in claim 11, wherein substeps i) to iii) are performed at a temperature of between 120° C. and 180° C.

13. The process as claimed in claim 11, wherein substep iii) is performed at a pressure of between 500 and 20 mbar and, more particularly, between 500 and 200.

14. The process as claimed in claim 11, wherein said substep iii) lasts between 30 minutes and 10 hours.

15. The process as claimed in claim 11, wherein, in substep ii), the polymerization inhibitor is chosen from 1,4-benzoquinone, 2-tert-butyl-1,4-benzoquinone, phenothiazine, 6-tert-butyl-2,4-xylenol, copper(II) dibutyldithiocarbamate, 1,1-diphenyl-2-picrylhydrazyl, 2,6-di-tert-butyl-p-cresol, 4-tert-butylpyrocatechol, 2,6-di-tert-butylphenol, hydroquinone, tert-butylhydroquinone and 4-methoxyphenol.

16. The process as claimed in claim 11, wherein, in substep ii), the esterification catalyst is an acid chosen from sulfuric, hydrochloric, phosphoric, nitric, hypophosphorous, methanesulfonic, para-toluenesulfonic and trifluoromethanesulfonic acids and acidic ion-exchange resins.
